(19) United States
(12) Patent Application Publication
(10) Pub. No.: US 2015/0289792 A1
(43) Pub. Date: Oct. 15, 2015

(54) LANCET ASSEMBLY AND LANCET STORAGE DEVICE

(71) Applicant: In Sang CHOI, Anyang-si Gyeonggi-do (KR)

(72) Inventor: In Sang Choi, Anyang-si, Gyeonggi-do (KR)

(21) Appl. No.: 14/437,336

(22) PCT Filed: Oct. 22, 2013
(86) PCT No.: PCT/KR2013/009410

§ 371 Date: Apr. 21, 2015

(30) Foreign Application Priority Data
Oct. 23, 2012 (KR) 10-2012-0117730
Oct. 23, 2012 (KR) 10-2012-0117732

(51) Int. Cl. A61B 5/151; A61B 5/15

(52) U.S. Cl. CPC A61B 5/15117; A61B 5/15186; A61B 5/150534

(57) ABSTRACT

A lancet assembly used in a lancing device, includes a launching device for advancing a lancet to be withdrawn to the outside and a lever for stopping and allowing the advance of the lancet or launching device, and includes: a lancet; a lancet body exposing the front of the lancet and coupled to the back of the lancet; and a protective cap enveloping the front of the lancet exposed from the lancet body and closely coupled to the lancet body. The rear end of the lancet body is coupled to the launching device. The lancet body and the protective cap are separated and coupled, allowing loading into the launching device. The lancet body is coupled to and separated from the launching device by an insertion protrusion and a joint, and the lancet body and the protective cap are recoupled by a central groove or protrusion formed in the protective cap.

### TECHNICAL FIELD

The present invention relates to a lancet assembly and a lancet storage device. More particularly, the present invention relates to a lancet assembly capable of protruding from a lancing device to measure blood glucose and implementing separation and reconnection of a protection cap and a lancet storage device allowing the lancet assembly to be stored and retrieved therefrom.

### BACKGROUND ART

A lancing device is generally configured to take blood and return to the original position thereof using elastic force of a spring in order to test blood glucose in a body.

Specifically, after a cap is removed from the lancing device, a separate lancet is inserted through an insertion hole. Then, a protection cap of the lancet is removed and the cap of the lancing device is reconnected. Thereby, when a button in the lancing device is pushed, the lance is moved down into a body by operation of the spring.

Thereafter, the cap of the lancing device removed, the protection cap of the lancet is reconnected, and then the lancet is removed from the insertion hole.

Accordingly, the lancing device and the lancet always need to be inconveniently carried together. In addition, it is inconvenient to manually insert the lancet into the insertion hole.

To address these problems, Korean Patent Application Publication No. 10-2009-0049505 discloses “LANCET DEVICE AND BLOOD-GATHERING METHOD USING THE SAME.” According to this document, a lancet device includes a lancet installation unit having an upper portion provided with a lancet installation groove for installing a disposable lancet provided with a protection cap-coupled lancet needle on one end of a lancet body, one side surface provided with an operation button, the other side surface provided with a depth adjustment lever for adjusting a insertion depth of the lancet needle into a skin, and a lower portion provided with a lancet removing button for separating the disposable lancet; a loading lever unit installed at a rear end of the lancet installation unit and provided with a loading lever operatively connected to the operation button and a loading operator, and an end cap unit installed at a front end of the lancet installation unit and provided with a through-hole allowing the lancet needle to momentarily protrude therethrough.

Korean Patent Application Publication No. 10-2009-0049505 discloses that the lancet device can be installed without the protection cap separated by causing the protection cap to be automatically removed from the disposable lancet in the step of loading the lancet, while conventional products requires the user to manually remove the protection cap from the disposable lancet to install the lancet device.

However, conventional lancet devices including the lancet device disclosed in Korean Patent Application Publication No. 10-2009-0049505 causes inconvenience to the user since the protection cap cannot be reconnected to the lancet once it is removed and thus the user needs to discard the lancet after coupling the protection cap to the front end of the lancet that has been used. Therefore, there is a need for a proper lancet capable of maintaining the protection cap to be separable from the lancet device after the protection cap is automatically reconnected, which has not been disclosed in the prior art.

In addition, for the conventional lancet devices, if the user uses a new lancet, the user needs to remove the old lancet and install the new lancet, which is inconvenient. Accordingly, there is a need for improvement of the lancet devices.

### DISCLOSURE

### Technical Problem

An object of the present invention is to an lancet assembly allowing a protection cap to be easily coupled to the lancet body in a lancing device after a lancet is withdrawn from the lancing device and used and allowing the lancet body to be readily coupled to and decoupled from a launcher.

Another object of the present invention is to provide a lancet storage device separably coupled to the lancing device and allowing multiple lancets to be continuously supplied to eliminate inconvenience of the user replacing the lancet every time a new lancet is used.

### Technical Solution

The objects of the present invention can be achieved by providing a lancet assembly used in a lancing device including a launcher configured to move a lancet forward to withdraw the lancet outside and a lever configured to allow and obstruct forward movement of the lancet or launcher, the lancet assembly comprising the lancet, a lancet body configured to expose a front of the lancet and coupled to a back of the lancet, and a protection cap configured to surround the front of the lancet exposed from the lancet body and coupled to the lancet body by closely contacting the lancet body, wherein a rear end of the lancet body is coupled to the launcher.

The lancet body is inserted into a front end of the launcher and coupled to the launcher, wherein the lancet body comprises an insertion projection inserted into the launcher with an edge there in close contact with the launcher, a connector formed in front of the insertion projection and having a smaller diameter than the insertion projection, and a rear close contact part formed in front of the connector to closely contact the front end of the launcher.

A diameter of a rear end of the insertion projection decreases as the rear end extends backward.

The lancet body includes a front close contact part configured to closely contact a rear end of the protection cap and a catch part arranged with a reduced diameter between the front close contact part and the rear close contact part.

The protection cap includes a neck configured to accommodate the lancet and a head arranged with an increased diameter in front of the neck.

According to an embodiment of the present invention, the lancet body and protection cap have circular lateral cross sections with respect to the lancet, wherein a center groove is concavely formed at a center of a front end of the protection cap.

Alternatively, the lancet body and protection cap have circular lateral cross sections with respect to the lancet, wherein a center projection is convexly formed at a center of a front end of the protection cap.

In another aspect of the present invention, provided herein is a lancet storage device configured to accommodate the lancet assembly according to any one of claims 1 to 6, the lancet storage device including a housing configured to store two or more items of the lancet assembly arranged in series, one side of the housing being open and provided with a withdrawer, an elastic part provided in the housing and configured to press the lancet assembly toward the withdrawer, and a loader arranged adjacent to the withdrawer and formed to be elastically deformable, the loader being configured to allow or obstruct withdrawal of the lancet assembly.

The elastic part includes a push plate configured to closely contact the lancet assembly and a push spring configured to press the push plate.

The housing is provided with a spring insertion part, the push spring being fittingly inserted into and supported by the spring insertion part.

The lancet body has a circular lateral cross section with respect to the lancet, wherein a distance from an end of the loader closely contacting the lancet body to an end of the withdrawer is less than a diameter of the lancet body.

The loader includes loading plate integrally connected to the housing in a cutaway form and configured to closely contact the lancet body and a loading projection formed on an outer side of the loading plate.

The lancet body is provided with an insertion projection protruding backward, wherein the housing is provided with an insertion groove formed in a direction of withdrawal of the lancet assembly to allow the insertion projection to be inserted thereinto.

The lancet storage device according to claim 9, wherein the lancet body includes a front close contact part configured to closely contact the rear end of the protection cap, a rear close contact part spaced apart from the front close contact part and having the same diameter as the front close contact part, and a catch part formed with a reduced diameter between the front close contact part and the rear close contact part, wherein the housing is provided with a guide projection inserted into the catch part and arrange in a direction of withdrawal of the lancet assembly.

### Advantageous Effects

According to an embodiment of the present invention, a lancet body and a protection cap are readily separated from and coupled to each other, and thus loading a launcher can be facilitated. In addition, the lancet body can be easily and safely coupled to and decoupled from the launcher by a insertion projection and a connector, and reconnection of the lancet body to the protection cap can be facilitated by a center groove or center projection formed on the protection cap.

According to an embodiment of the present invention, multiple lancet assemblies can be accommodated in and withdrawn from a lancet storage device, and therefore inconvenience of replacing lancets every time a lancet is used can be eliminated. In addition, by providing a guide projection and an insertion groove, the lancet assemblies can be stably withdrawn and moved to the lancet device, and the lancet can be stably used.

### DESCRIPTION OF DRAWINGS

FIG. 1 is a plan view illustrating a lancet assembly coupled to a lancing device according to an embodiment of the present invention.

FIG. 2 is a perspective view illustrating the lancet assembly shown in FIG. 1.

FIG. 3 is an exploded view illustrating components of the lancet assembly shown in FIG. 2.

FIG. 4 is an exploded view illustrating components of a lancet assembly according to another embodiment of the present invention.

FIG. 5 is a view illustrating use of a lancet storage device according to another embodiment of the present invention.

FIG. 6 is a front view illustrating the lancet storage device shown in FIG. 5.

FIG. 7 is a side view illustrating the lancet storage device shown in FIG. 5.

FIG. 8 is a bottom view illustrating the lancet storage device shown in FIG. 5.

FIG. 9 is a perspective view illustrating the lancet storage device shown in FIG. 5. with some parts omitted.

FIG. 10 is a front view illustrating the lancet storage device shown in FIG. 9.

Description of Reference Numerals of Main Parts in the Drawings

1: Lancet assembly 10: Lancet
20: Lancet body 21: Front close contact part
22: Catch part 23: Rear close contact part
24: Connector 25: Insertion projection
30: Protection cap 31: Head
32: Neck 33: Center groove
34: Center projection 2: Lancet storage device
100: Housing 110: With drawer
120: Spring insertion part 130: Insertion groove
140: Guide projection 200: Elastic part
210: Push plate 220: Push spring
300: Loader 310: Loading plate
320: L projection

### BEST MODE

Reference will now be made in detail to the preferred embodiments of the present invention, examples of which are illustrated in the accompanying drawings. In describing the present invention, descriptions of well-known technologies may be omitted in order not to obscure the gist of the present invention.

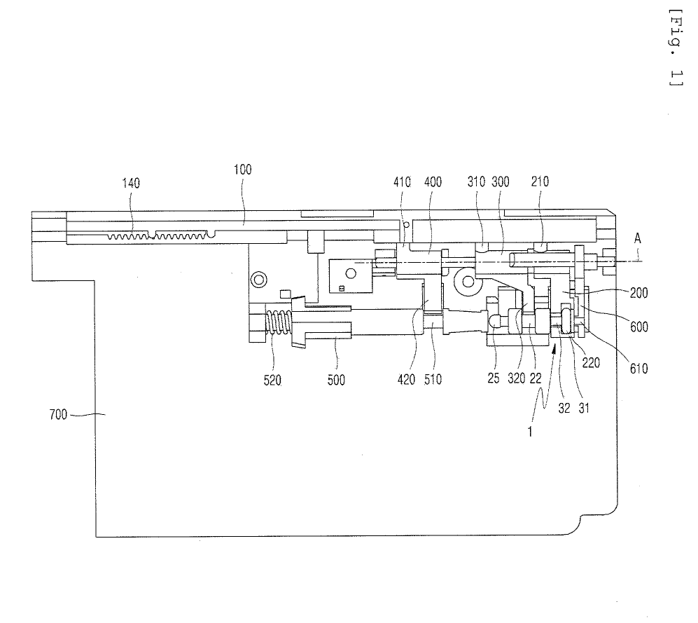

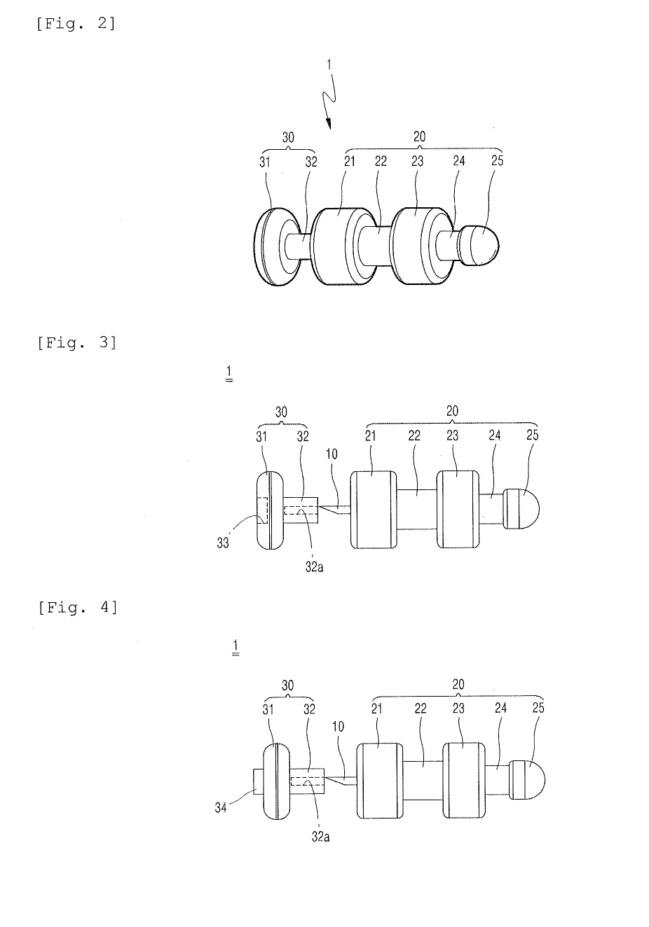

FIG. 1 is a plan view illustrating a lancet assembly 1 coupled to a lancing device according to an embodiment of the present invention, FIG. 2 is a perspective view illustrating the lancet assembly 1 shown in FIG. 1, and FIG. 3 is an exploded view illustrating the lancet assembly 1 shown in FIG. 2 FIG. 3.

In the description given below, directions are defined based on FIG. 1. In FIG. 1, the forward direction points to the right side of the figure, the backward direction points to the left side of the figure. The upward direction points to the viewer of the figure, and the downward direction points down into the figure.

In this invention, a lancet 10, a lancet body 20 and a protection cap 30 are coupled to construct a lancet assembly 1. The sharp lancet 10 is coupled to the front of the lancet body 20, and the protection cap 30, which accommodates the lancet 10 and prevents the lancet 10 from being exposed outside, is coupled to the lancet body 20. The protection cap 30 is formed such that the back of the protection cap 30 has a smaller diameter than the front thereof, and the lancet body 20 is formed such that the middle part (the catch part 22) of the lancet body 20 has a smaller diameter than the other part thereof.

Before a detailed description is given of the lancet assembly 1, a lancing device employing the lancet assembly 1 will be described first.

According to an embodiment, the lancing device is configured to take blood to test blood glucose. In particular, the lancing device allows for execution of a series of operations including separating the protection cap 30 from the lancet body 20 coupled to the lancet 10, launching the lancet 10 and reconnecting the protection cap 30 to the lancet body 20. The lancing device may be configured to have the same structure and operations as those of a typical lancing device except the aforementioned operations.

The lancing device includes a cam plate 100, a cap lever 200, a collet lever 300, a launch lever 400 and a fixing lever 600.

The cam plate 100 is generally formed in a flat and long plate shape. Formed on one side surface of the cam plate 100 are a first catch part, a second catch part, a third catch part and a rack gear 140. In forming the lancing device, the cam plate 100 is positioned to one side of a baseplate 700 and perpendicularly connected to the baseplate 700.

With the cam plate 100 connected to the baseplate 700, the first catch part, the second catch part, the third catch part and the rack gear 140 are arranged on the inner surface of the cam plate 100. As the cam plate 100 slidably moves back and forth according to rotation of a drive gear (not shown) engaged with the rack gear 140, the cap lever 200, the collet lever 3000 and the launch lever 400 rotates in operative connection with each other. Thereby, the lancet body 20 and the protection cap 30 are separated and coupled, and a launcher 500 is controlled.

The first catch part, the second catch part and the third catch part are arranged in order from the front to back of the cam plate 100. The catch parts are formed to guide movements of the cap lever 200, the collet lever 300 and the launch lever 400, respectively. The first catch part, the second catch part and the third catch part may be concavely formed in a groove shape.

The cap lever 200 is coupled to the cam plate 100 so as to rotate about a pivot axis A arranged parallel to the movement direction of the cam plate 100. The cap lever 200 includes a first projection 210, a cap arm 220 and a pivot slot part. The cap lever 200 cannot fully rotate about the pivot axis A, but can rotate downward within a predetermined range in a reciprocating manner.

The first projection 210 is inserted into the first catch part to slidably move therein. Thereby, the cap lever 200 rotates about the pivot axis A within a predetermined range of angle.

The cap arm 220 is formed approximately on the opposite side of a part of the cap lever 200 that is connected to the pivot axis A with respect to the side of the part of the cap lever 200 on which the first projection 210 is formed. The cap arm 220 is formed to be coupled to the protection cap 30 by surrounding a portion of the protection cap 30. As the cap arm 220 is provided with a groove to surround a portion of the protection cap 30, the protection cap 30 is caught by the cap arm 220.

The pivot slot part extends toward the collet lever 300 to allow a second projection 310, which will be described later, to be inserted thereinto. Accordingly, the pivot slot part has a long slot extending in the movement direction of the cam plate 100, and the second projection 310 is inserted in this slot.

The fixing lever 600 is connected to rotate about a pivot axis A and to move in the axial direction of the pivot axis A. In addition, the fixing lever 600 is elastically supported against the cap lever 200. One side of the fixing lever 600 is provided with a fixing projection 610. The fixing projection 610 is coupled to the cap arm 220 and the protection cap 30. Specifically, when the fixing lever 600 is spaced apart from the cap lever 200, the fixing projection 610 is also spaced apart from the cap arm 220 and the protection cap 30. When the fixing lever 600 closely contacts the cap lever 200, the fixing projection 610 is inserted into the cap arm 220 through a grove formed in the cap arm 220 and coupled to the protection cap 30.

An end of the fixing projection 610 may be formed in a projection shape as shown in FIG. 1. Alternatively, the end of the fixing projection 610 may be formed in a concave groove shape. The fixing projection 610 is coupled to a center groove 33 or center projection 34 of the protection cap 30, as will be described below. When the end of the fixing projection 610 is formed in a projection shape, the end is inserted into the center groove 33 and coupled to the protection cap 30. When the end of the fixing projection 610 is formed in a groove shape, the end is coupled to the protection cap 30 by accommodating the center projection 34.

The collet lever 300 is connected to rotate about the pivot axis A and is provided with a second projection 310 and a body arm 320. The collet lever 300 cannot fully rotate about the pivot axis A, but can rotate downward within a predetermined range in a reciprocating manner.

The second projection 310 is inserted into the second catch part to slidably move therein. Thereby, the collet lever 300 rotates about the pivot axis A within a predetermined range of angle.

The body arm 320 is formed approximately on the opposite side of a part of the collet lever 300 that is connected to the pivot axis A with respect to the side of the part of the collet lever 300 on which the second projection 310 is formed. The body arm 320 is formed to catch the lancet body 20. As will be described later, the lancet body 20 has a part (a catch part 22) with a reduced diameter. An end of the body arm 320 is inserted into this part and caught by the lancet body 20.

The launch lever 400 is connected to rotate about the pivot axis A and is provided with a third projection 410 and a launch arm 420. The launch lever 400 cannot fully rotate about the pivot axis A, but can rotate downward within a predetermined range in a reciprocating manner.

The third projection 410 is inserted into the third catch part to slidably move therein. Thereby, the launch lever 400 rotates about the pivot axis A within a predetermined range of angle.

The launch arm 420 is formed approximately on the opposite side of a part of the cap lever 200 that is connected to the pivot axis A with respect to the side of the part of the cap lever 200 on which the third projection 410 is formed. The launch arm 420 is formed to catch a launcher 500.

The launcher 500 is coupled to the lancet body 20 to move forward. To this end, the rear end of the launcher 500 is provided with an elastic member 520 such as a coil spring which produces elastic force when compressed. When the means to stop forward movement of the launcher 500 is excluded, the launcher 500 is moved forward by the elastic force.

The front end of the launcher 500 is formed to allow the rear end of the lancet body 20 to be coupled thereto in an inserting manner. Specifically, the front end of the launcher 500 is elastically deformable (and may be formed of a material such as plastics or metal). In particular, a part of the front end of the launcher 500 is cut away to allow the rear end of the lancet body 20 to be fixed (by friction) by being naturally inserted into the front end and to be decoupled from the launcher 500.

One side 510 of the launcher 500 is formed such that the diameter thereof decreases. Thereby, the launch arm 420 is inserted into the launcher 500 and caught by the one side 510.

The lancet body 20 includes an insertion projection 25, a connector 24, a rear close contact part 23, a catch part 22 and a front close contact part 21, which are arrange in a direction pointing from back to front. The lateral cross section of the lancet body 20 with respect to the lancet 10 has a circular shape.

The insertion projection 25 is inserted into the front end of the launcher such that the edge of the insertion projection 25 closely contacts the launcher. The diameter of the rear end of the insertion projection 25 decrease as the rear end extends backward. That is, as shown in FIGS. 2 and 3, the front part of the insertion projection 25 is cylindrically formed, and the rear part thereof is formed in a semi-spherical shape and the diameter of the rear part decreases as the rear part extends backward. Thereby, the insertion projection 25 can be easily inserted into the front end of the launcher and supported by the launcher by stably and closely contacting the interior of the front end of the launcher.

The connector 24 is formed at the front of the insertion projection 25 and has a smaller diameter than the insertion projection 25. That is, when the insertion projection 25 is inserted into the front end of the launcher, the launcher closely contacts the outer circumferential surface of the insertion projection 25, but does not closely contacts the connector 24. Thereby, the front end portion of the launcher strongly and closely contact the insertion projection 25, and the diameter of the front end portion is reduced at a position where the connector 24 is located. Thereby, the front end of the insertion projection 25 is caught and thus the lancet body 20 can be stably coupled to the launcher.

The rear close contact part 23 is formed at the front of the connector 24 to closely contact the front end of the launcher. That is, when the insertion projection 25 is inserted into the front end of the launcher, the front end of the launcher moves until it closely contacts the rear close contact part 23.

The front close contact part 21 forms the foremost part of the lancet body 20 and closely contacts the rear end of the protection cap 30. To smoothly move in the lancing device, the front close contact part 21 preferably has the same diameter as that of the rear close contact part 23.

The diameter of the catch part 22 decreases between the front close contact part 21 and the rear close contact part 23. As the body arm 320 is caught by the catch part 22, movement of the lancet body 20 is restricted by movement of the collet lever 300. That is, the collet lever 300 and the lancet body 20 are coupled and decoupled according to rotation of the collet lever 300. As the collet lever 300 moves backward, the lancet body 20 (and the lancet 10) is decoupled from the protection cap 30, and the rear end of the lancet body 20 is coupled to the front end of the launcher 500.

The protection cap 30 includes a neck 32 and a head 31. The lateral cross section of the protection cap 30 with respect to the lancet 10 has a circular shape.

The neck 32 is provided with a groove 32a in the center thereof to accommodate the lancet 10. The diameter of the head 31 increases at the front of the neck 32.

As shown in FIG. 3, the center of the front end of the protection cap 30, i.e., the front end of the head 31 may be provided with a center groove 33. The fixing projection 610 (with the end part formed in a projection shape) described above is fitted into the center groove 33. When the fixing projection 610 is inserted into the center groove 33 with the protection cap 30 accommodated in the cap arm 220, the cap lever 200, the fixing lever 600 and the protection cap 30 are stably coupled to each other without spacing therebetween.

As shown in FIG. 4, center projection 34 may be convexly formed at the center of the front end of the protection cap 30, i.e., the front end of the head 31. The fixing projection 610 (with the end part formed in a projection shape) described above is fitted into the center projection 34. When the fixing projection 610 and the center projection 34 are coupled with the protection cap 30 accommodated in the cap arm 220, the cap lever 200, the fixing lever 600 and the protection cap 30 are stably coupled without spacing therebetween.

That is, by providing the center groove 33 or the center projection 34 to the protection cap 30, the protection cap 30 is stably accommodated and coupled to the cap lever 200 when the protection cap 30 is separated from the lancet body 20 and rotates along with the cap lever 200 and the fixing lever 600. When the protection cap 30 is re-coupled to the lancet body 20, the center of the protection cap 30 is aligned with the center of the lancet body 20 and thus the lancet 10 is smoothly fitted into the protection cap 30 since there is no spacing between the protection cap 30 and the lancet body 20.

As described above, the lancet assembly 1 is divided into the lancet body 20 including the insertion projection 25, the connector 24, the rear close contact part 23, the catch part 22 and the front close contact part 21 and the protection cap 30 including the neck 32 and the head 31. Thereby, the lancet body 20 can be separated backward from the cap lever by interoperation of the cam plate 100, the cap lever 200 and the collet lever 300, and coupled to the launcher, and the lancet body 20 can be re-coupled to the protection cap 30. In addition, the protection cap 30 is provided with the center groove 33 or the center projection 34, and thus the protection cap 30 is stably supported by and smoothly re-coupled to the cap lever.

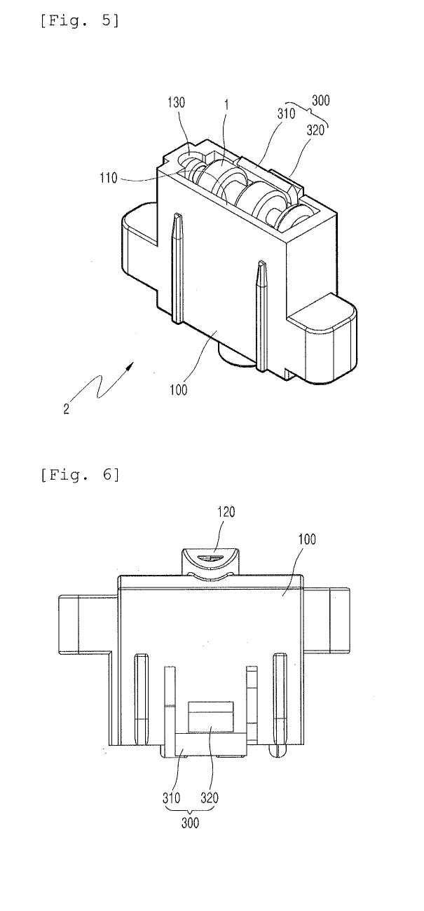

FIG. 5 is a view illustrating use of a lancet storage device 2 according to another embodiment of the present invention.

The lancet storage device 2 can be used by being coupled to a lancing device for measurement of blood glucose. The lancet 10, particularly a stack of multiple lancet assemblies 1 is stored in the lancet storage device 2.

The lancet storage device 2 is used by being coupled to the lancing device. That is, as the lancet storage device 2 is coupled to the lancing device, the lancet assembly 1 stored in the lancet storage device 2 moves toward the lancing device. As the lancet 10 is withdrawn from the lancing device, blood is taken by the lancet 10.

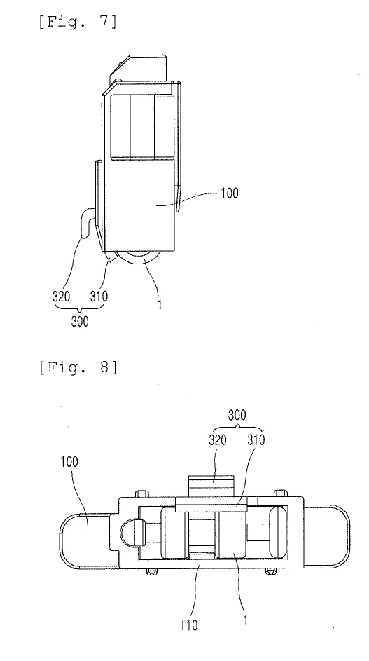

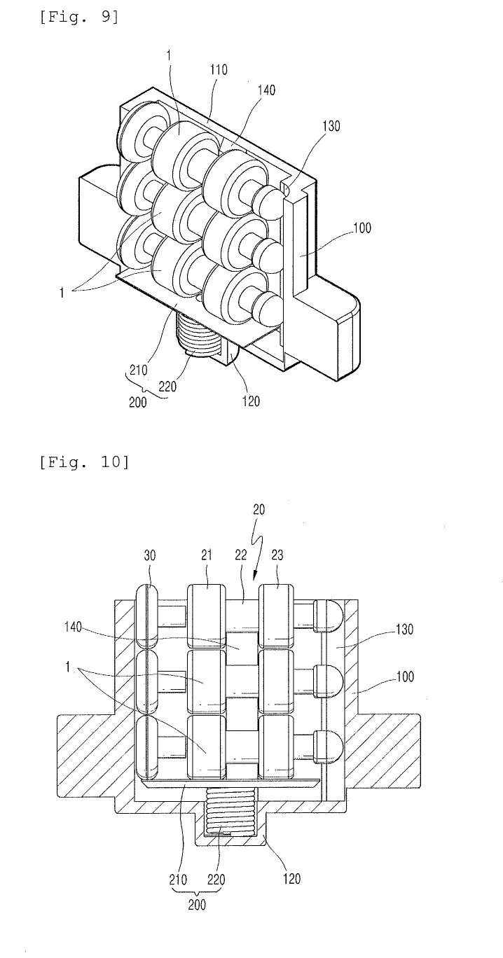

FIG. 6 is a front view illustrating the lancet storage device 2 shown in FIG. 5, FIG. 7 is a side view illustrating the lancet storage device 2 shown in FIG. 5, and FIG. 8 is a bottom view illustrating the lancet storage device 2 shown in FIG. 5. FIG. 9 is a perspective view illustrating the lancet storage device 2 shown in FIG. 5, and FIG. 10 is a front view illustrating the lancet storage device 2 shown in FIG. 9.

The lancet storage device 2 includes a housing 100, an elastic part 200 and a loader 300.

One side of the housing 100 is open and provided with a withdrawer 110. Formed inside the housing 100 are an insertion groove 130 and a guide projection 140. A spring insertion part 120 is formed in the housing 100 on the side of the housing 100 facing the withdrawer 110.

The cross section of the housing 100 is approximately rectangular. Thereby, the withdrawer 110 is also rectangular. The interior of the housing 100 fits to slidable movement of the lancet assembly 1. Thereby, the long distance between inner walls of the housing 100 corresponds to the length of the lancet assembly 1 and the short distance between the inner wall corresponds to the width of the lancet assembly 1.

The housing 100 may be formed of metal, or a synthetic resin such as plastics. Preferably the housing 100 is formed of a synthetic resin for manufacturing convenience.

The housing 100 has an inner depth allowing two or more the lancet assembles 1 to be stored therein. The housing 100 may be formed to store three lancet assemblies 1, as shown in FIG. 9, or more lancet assemblies.

The insertion groove 130 is formed to have a semi-circular cross section to allow the insertion projection 25 to fit thereto, and extends in the withdrawing direction of the lancet assembly 1. Thereby, when the lancet assembly 1 is withdrawn from the housing 100, the insertion projection 25 is guided by the insertion groove 130, and stable withdrawing is implemented without spacing of the lancet assembly 1.

The guide projection 140 protrudes from the inner surface from the housing 100, and extends in the withdrawing direction of the lancet assembly 1 as the insertion groove 130 does. When the lancet assembly 1 is stored in the housing 100, the guide projection 140 is positioned between the front close contact part 21 and the rear close contact part 23. That is, the guide projection 140 faces and closely contacts the catch part 22. Thereby, when the lancet assembly 1 is withdrawn, the lancet body 20 is guided by the guide projection 140, and more stable withdrawing is implemented without spacing of the lancet assembly 1.

In the housing 100, the spring insertion part 120 is formed on the opposite side to the side on which the withdrawer 110 is formed, and one side end of a push spring 220 of the elastic part 200 is inserted into and supported by the spring insertion part 120.

The elastic part 200 is provided in the housing 100 and configured to apply force to the lancet assembly 1 toward the withdrawer 110. To this end, the elastic part 200 includes the push plate 210 and a push spring 220.

The push plate 210 has a flat plate shape, and approximately corresponds to the area of the inner space of the housing 100. When the lancet assembly 1 is stored in the housing 100, the push plate 210 closely contacts a lancet assembly 1 positioned at the innermost side.

The push spring 220 is installed such that one side end thereof closely contacts the push plate 210, and the other side end thereof is inserted into the spring insertion part 120. The push spring 220 may be formed in the shape of a common coil spring. The push spring 220 produces elastic force to press the innermost lancet assembly to the withdrawer 110 when the lancet assemblies 1 are stored in the housing 100.

The loader 300 is arranged adjacent to the withdrawer 110 of the housing 100. The loader 300 obstructs or allow withdrawal of the lancet assembly 1 from the housing 100. To this end, the loader 300 includes a loading plate 310 integrally connected to the housing 100 in a cutaway form and a loading projection 320 formed on the outer side of the loading plate 310.

The loading plate 310, which is arranged in the housing 100 in a cutaway form, approximately forms the same plane as that of the sidewall of the housing 100, or is curved toward the lancet assembly 1 as it extends to the end thereof. In this case, the distance from the end of the loading plate 310 to the end of the withdrawer 110 facing the loading plate 310 is less than the diameter of the lancet body 20. Thereby, in usual cases, the lancet assembly 1 is caught by the loading plate 310 and obstructed from being withdrawn from the housing 100. When the loading plate 310 is curved outward, the lancet assembly 1 is withdrawn out of the housing 100.

The loading projection 320 is integrally formed in the shape of a bending projection on the exterior of the loading plate 310, as shown in FIG. 7. When the lancet storage device 2, which is configured to be coupled to the lancing device, is coupled to the lancing device, the loading projection 320 is caught by one side of the lancing device and pulled outward. At this time, the loading plate 310 is also bent outward, thus cannot obstruct withdrawal of the lancet assembly 1 from the housing 100, and the lancet assembly 1 is withdrawn by pressing force of the elastic part 200 applied thereto.

After formation of the lancet storage device 2 is completed, the lancet assembly 1 can be readily inserted into the housing 100 by pulling the loading projection 320 outward.

As such, the lancet storage device 2 is configured such that multiple lancet assemblies 1 are accommodated in the lancet storage device 2. Therefore, the lancets do not need to be replaced every time a new lancet is used. In addition, the guide projection 140 and the insertion groove 130 may ensure stable withdrawal of the lancet assemblies 1, stable movement of the lancet assemblies 1 to the lancing device and stable use of the lancet.

It will be apparent to those skilled in the art that various modifications and variations can be made in the present invention without departing from the spirit or scope of the invention. Thus, it is intended that the present invention covers the modifications and variations of this invention provided they come within the scope of the appended claims and their equivalents.

### INDUSTRIAL APPLICABILITY

According to an embodiment of a lancet assembly and a lancet storage device of the present invention, the lancet assembly allows a lancet body to be readily coupled to and decoupled from a launcher and multiple lancets to be continuously supplied. Thereby, the lancet storage device eliminates inconvenient of the user replacing lancets every time a lancet is used. As the present invention overcomes the limit of the prior art, the invention is applicable in the related art and a device to which the present invention is applied is very likely to come to the market. Further, since the present invention can be explicitly practiced in reality, it is industrially available.

1. A lancet assembly used in a lancing device including a launcher configured to move a lancet forward to withdraw the lancet outside and a lever configured to allow and obstruct forward movement of the lancet or launcher, the lancet assembly comprising:
the lancet;
a lancet body configured to expose a front of the lancet and coupled to a back of the lancet; and
a protection cap configured to surround the front of the lancet exposed from the lancet body and coupled to the lancet body by closely contacting the lancet body,
wherein a rear end of the lancet body is coupled to the launcher.

2. The lancet assembly according to claim 1, wherein the lancet body is inserted into a front end of the launcher and coupled to the launcher,
wherein the lancet body comprises an insertion projection inserted into the launcher with an edge there in close contact with the launcher, a connector formed in front of the insertion projection and having a smaller diameter than the insertion projection, and a rear close contact part formed in front of the connector to closely contact the front end of the launcher.

3. The lancet assembly according to claim 2, wherein a diameter of a rear end of the insertion projection decreases as the rear end extends backward.

4. The lancet assembly according to claim 2, wherein the lancet body comprises a front close contact part configured to closely contact a rear end of the protection cap and a catch part arranged with a reduced diameter between the front close contact part and the rear close contact part.

5. The lancet assembly according to claim 1, wherein the protection cap comprises a neck configured to accommodate the lancet and a head arranged with an increased diameter in front of the neck.

6. The lancet assembly according to claim 1, wherein the lancet body and the protection cap have circular lateral cross sections with respect to the lancet,
wherein a center groove is concavely formed at a center of a front end of the protection cap.

7. The lancet assembly according to claim 1, wherein the lancet body and the protection cap have circular lateral cross sections with respect to the lancet,
wherein a center projection is convexly formed at a center of a front end of the protection cap.

8. A lancet storage device configured to accommodate the lancet assembly according to claim 1, the lancet storage device comprising:
a housing configured to store two or more items of the lancet assembly arranged in series, one side of the housing being open and provided with a withdrawer;
an elastic part provided in the housing and configured to press the lancet assembly toward the withdrawer; and
a loader arranged adjacent to the withdrawer and formed to be elastically deformable, the loader being configured to allow or obstruct withdrawal of the lancet assembly.

9. The lancet storage device according to claim 8, wherein the elastic part comprises a push plate configured to closely contact the lancet assembly and a push spring configured to press the push plate.

10. The lancet storage device according to claim 9, wherein the housing is provided with a spring insertion part, the push spring being fittingly inserted into and supported by the spring insertion part.

11. The lancet storage device according to claim 8, wherein the lancet body has a circular lateral cross section with respect to the lancet,
wherein a distance from an end of the loader closely contacting the lancet body to an end of the withdrawer is less than a diameter of the lancet body.

12. The lancet storage device according to claim 10, wherein the loader comprises loading plate integrally connected to the housing in a cutaway form and configured to closely contact the lancet body and a loading projection formed on an outer side of the loading plate.

13. The lancet storage device according to claim 8, wherein the lancet body is provided with an insertion projection protruding backward,
wherein the housing is provided with an insertion groove formed in a direction of withdrawal of the lancet assembly to allow the insertion projection to be inserted thereinto.

14. The lancet storage device according to claim 9, wherein the lancet body comprises a front close contact part configured to closely contact the rear end of the protection cap, a rear close contact part spaced apart from the front close contact part and having the same diameter as the front close contact part, and a catch part formed with a reduced diameter between the front close contact part and the rear close contact part,
wherein the housing is provided with a guide projection inserted into the catch part and arrange in a direction of withdrawal of the lancet assembly.
